(19) United States
(12) Patent Application Publication
(10) Pub. No.: US 2024/0000327 A1
(43) Pub. Date: Jan. 4, 2024

(54) DEVICES AND METHODS FOR PREDICTING A HEART RATE VARIABILITY PARAMETER

(71) Applicant: Huma Therapeutics Limited, London (GB)

(72) Inventors: Davide Morelli, London (GB); Leonardo Bartoloni, London (GB)

(21) Appl. No.: 18/265,165

(22) PCT Filed: Dec. 3, 2021
(86) PCT No.: PCT/EP2021/084284

§ 371 Date: Jun. 2, 2023

(30) Foreign Application Priority Data
Dec. 4, 2020 (GB) 2019174.8

(51) Int. Cl. A61B 5/024; A61B 5/00; G06F 17/11

(52) U.S. Cl. CPC A61B 5/02405; A61B 5/681; A61B 5/0022; A61B 5/7221; A61B 5/742; G06F 17/11

(57) ABSTRACT

A device for predicting a heart rate variability parameter for a user. The device including one or more processing modules configured to receive a set of heart rate measurements for a user obtained over a period of time with data indicating a time at which each measurement was obtained during the period of time, process the heart rate measurements to calculate a heart rate variability parameter over the period of time and to estimate whether the set is sufficient for calculating the heart rate variability parameter over the period of time, and to form an uncertainty score representing the result of that estimation, determine whether the heart rate variability parameter is valid by comparing the uncertainty score to a predetermined threshold, and output, in response to a determination that the heart rate variability parameter is valid, the heart rate variability parameter and a representation of the uncertainty score.

### RELATED APPLICATION

This application claims priority to and the benefit of GB application number GB 2019174.8 filed Dec. 4, 2020, the disclosure of which is incorporated herein in its entirety.

### FIELD OF THE INVENTION

Embodiments of the invention relate to devices and methods for predicting a heart rate variability parameter for a user.

### BACKGROUND

Cardiovascular disease is a term that is commonly used to describe a range of conditions that affect the heart or blood circulation of a patient. It is responsible for over 30% of global deaths and is a cause of mounting concern for both medical professionals and members of the general public. Methods for monitoring general cardiac health to predict, detect and mitigate the onset of cardiovascular disease are therefore becoming increasingly more important.

A number of parameters can be useful to monitor in assessing the cardiac health of an individual. Heart rate variability (HRV) is an example of such a parameter and is a measure of the variation in time intervals between consecutive heartbeats. Various features of HRV may provide a more specific characteristic of cardiac health. An example of such a feature is the standard deviation of the inter-beat interval between heart beats that is collected over a 24 hour period (SDNN24). Patients suffering from cardiovascular disease are reported to have significantly lower SDNN24 readings than those of healthy individuals.

A traditional method of obtaining an SDNN24 reading requires a patient to wear a Holter monitor for a 24 hour period. A Holter monitor is an apparatus comprising a recording device that is connected to a number of electrodes that are distributed over and adhered to patient's chest. The wearing of this monitor for a 24 hour period is an invasive procedure and is therefore disruptive to the everyday life of the patient. As a result of the invasive nature of its testing procedure, SDNN24 is not performed routinely on the general population, and is instead most frequently only identified after a patient has developed symptoms of cardiovascular disease and approaches a medical professional.

### SUMMARY

In an aspect, embodiments of the invention relate to a device for predicting a heart rate variability parameter for a user, the device comprising one or more processing modules and being configured to: receive a set of heart rate measurements for a user obtained over a period of time, together with data indicating a time at which each heart rate measurement was obtained during the period of time; process the set of heart rate measurements to calculate a heart rate variability parameter over the period of time; further process the set of heart rate measurements to estimate whether the set is sufficient for calculating the heart rate variability parameter over the period of time, and to form an uncertainty score representing the result of that estimation; determine whether the heart rate variability parameter is valid by comparing the uncertainty score to a predetermined threshold; and output, in response to a determination that the heart rate variability parameter is valid, the heart rate variability parameter and a representation of the uncertainty score.

One or more of the following features may be included. The set of heart rate measurements may be received from a second device that is external to the first device, the second device comprising a sensor for recording a plurality of heart rate measurements over time.

The second device may be a wrist-worn fitness tracker.

The set of heart rate measurements may be received directly from the second device via a wired or wireless connection.

The set of heart rate measurements may be received via a network that stores data recorded by the second device.

The heart rate variability parameter may be determined to be valid if the uncertainty score is below the predetermined threshold.

The set of heart rate measurements may be discarded if it is determined that the heart rate variability parameter is not valid.

The heart rate variability parameter may be the standard deviation of the inter-beat interval of sinus beats.

The period of time may be a 24 hour time period.

The heart rate measurements that are used to calculate the heart rate variability parameter may be in the ultra-low frequency spectrum.

The heart rate variability parameter may be output to a further calculation unit that determines whether the value of that parameter is indicative of cardiovascular disease.

The output may comprise demonstrating the heart rate variability and the representation of the uncertainty score on a display of the first device.

An estimated value of the heart rate variability parameter may be calculated using the following calculation:

SDNNₑₛₜ=q+mSDNN_{LF}
wherein SDNN_{LF} is defined by:

$$S D N {N}_{L F} = 60000 \sqrt{{\sum }_{k = - 2}^{2} {\vert {f}_{k} \vert}^{2} {β}_{k}}$$

wherein fₖ is a calculated best fit parameter and βₖ is a vector defined as [1, 1, 0, 1, 1].

The uncertainty score may be formed using the following calculation:

$$Δ S D N {N}_{e s t} = \frac{m}{S D N {N}_{L F}} {\sum }_{k = - 2}^{2} {α}_{k} Δ {θ}_{k}$$

wherein αₖ is a solution of a system that indicates which linear combination of eigenvectors will produce the vector βₖ and Δθₖ is a variance of the k^{th} value of a parameter θ, and is defined by:

$$Δ {θ}_{k} = \frac{{Δ}_{0}}{\sqrt{{λ}_{k}}}$$

wherein λₖ is an eigenvalue of a matrix S corresponding to a normalised eigenvector vₖ, and Δ₀ is a constant expressing how much information each heart rate measurement contributed to the uncertainty score.

In another aspect, embodiments of the invention relate to a method for predicting a heart rate variability parameter for a user using a first device. The method includes receiving a set of heart rate measurements for a user obtained over a period of time, together with data indicating a time at which each heart rate measurement was obtained; processing the set of heart rate measurements to calculate a heart rate variability parameter over the period of time; processing the set of heart rate measurements to estimate whether the set is sufficient for calculating the heart rate variability parameter over the period of time and forming an uncertainty score representing the result of that estimation; determining whether the heart rate variability parameter is valid by comparing the uncertainty score to a predetermined threshold; and outputting, in response to a determination that the heart rate variability parameter is valid, the heart rate variability parameter and a representation of the uncertainty score.

### BRIEF DESCRIPTION OF DRAWINGS

Embodiments of the present invention will now be described by way of example with reference to the accompanying drawings. In the drawings:

FIG. 1 illustrates a system for monitoring a heart rate variability parameter of a user;

FIG. 2 illustrates a method for calculating a heart rate variability parameter and an uncertainty score for a user; and

FIG. 3 illustrates a method for monitoring the heart rate variability of a user, using the system illustrated in FIG. 1.

### DETAILED DESCRIPTION

As discussed above, a number of parameters can be useful to monitor in assessing the cardiac health of an individual. Heart rate variability (HRV) is an example of such a parameter and is a measure of the variation in time intervals between consecutive heartbeats. There are various features of HRV that may provide a more specific characteristic of cardiac health; an example of such a feature is SDNN24. SDNN24 is the standard deviation of the inter-beat interval between heart beats that is collected over a 24 hour period. Patients suffering from cardiovascular disease are reported to have significantly lower SDNN24 readings than those of healthy individuals.

However, methods of obtaining an SDNN24 reading can require a patient to wear a monitor (e.g., Holter monitor) for a 24 hour period. Due to the invasive nature of its testing procedure, SDNN24 is typically not performed on the general population routinely, and is instead most frequently only identified after a patient has developed symptoms of cardiovascular disease and approaches a medical professional.

As such, there is a need for a non-invasive, continuous measurement of heart rate variability features such as SDNN24 from which early warnings of cardiovascular disease can be derived. These early warnings may enable preventative actions to be taken before the patient begins experiencing symptoms of cardiovascular disease.

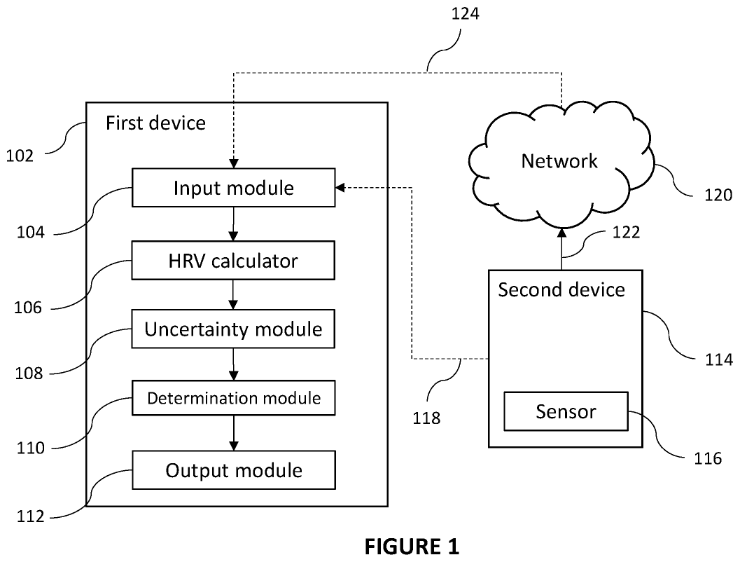

FIG. 1 illustrates a system for monitoring a heart rate variability parameter of a user. The system comprises a first device 102 which has a plurality of processing modules. The processing modules may comprise one or more data processing integrated circuits and/or one or more graphics processing integrated circuits. The processing modules may alternatively be implemented in software. Each processing module illustrated in FIG. 1 is configured to perform a different function. The processing modules comprise an input module 104, a HRV parameter calculator 106, an uncertainty calculation module 108, a determination module 110 and an output module 112. In FIG. 1, each of the modules 104-112 are illustrated as separate entities. However, it would be appreciated by a skilled person that in an alternative example of the invention one or modules may be combined within the same unit. In one example, the HRV parameter calculator 106 and the uncertainty calculation module 108 may be comprised within the same unit. The first device may comprise processing modules in addition to those that are illustrated in FIG. 1.

The first device 102 is connected to a second device 114. The second device 114 comprises a sensor 116. The second device may further comprise a memory for storing data that is recorded by the sensor 116. In one example the first device 102 is directly connected to the second device 114 by a first connection 118. The first connection 118 may be either a wired or a wireless connection. In another example which may be combined with the first example, the second device is connected to a network 120 by a connection 122. The connection 122 is a wireless connection. The first device 102 may also be connected to the network 120 by a wireless connection 124. Thus, the first device 102 may be able to receive data from the second device 114 via the network 120 by means of a wireless connection 124. The network 120 is configured to store data that is recorded by the sensor 116 of the second device 114. That is, the network 120 may be in communication with a cloud-based memory (e.g., server) for storing data recorded by sensor 116.

The second device 114 comprises a heart rate monitor. The second device may be a fitness tracker. The fitness tracker may be a commercially available fitness tracker. In one example, the second device 114 is a wrist-worn fitness tracker. In another example, the second device 114 is a chest-worn fitness tracker. The second device may be configured to obtain heart rate measurements from any alternative location on the body of a user. The sensor 116 is configured to record a plurality of heart rate measurements of a user over time. The heart rate of a user is defined as the number of beats per minute of the user's heart. Examples of types of sensor that may be used to measure the heart rate of a user are electrocardiogram (ECG) and photoplethysmography (PPG) sensors. It would be appreciated that other types of sensor may alternatively be used. The sensor 116 or second device 114 in general may be configured to obtain other physiological measurements from a user such as breathing rate, body temperature or blood pressure.

The data recorded by the second device 114 and/or the network 120 includes heart rate measurements, as well as an indication of the time at which the heart rate measurements were recorded and/or the frequency of measurements within a predefined time period.

The first device 102 may further comprise, at least, a display and storage medium such as memory that may be implemented on one or more integrated circuits and/or on a hard drive. The first device may comprise one or more additional processors, which may comprise one or more data processing integrated circuits and/or one or more graphics processing integrated circuits. The first device 102 may be a smartphone. An advantage of the device being a smartphone is that most individuals have access to such a device; accordingly, users may be able to obtain heart rate variability parameters in domestic settings and at regular intervals to monitor health. In an example where the first device 102 is a smart phone, the processing modules 104-112 may be implemented in software and comprised within an application that is run on the device. The application may be configured to access the second device 114, the network 120 or and the memory of the first device. The display of the first device may also be a user interface, such as the screen of a smartphone device. Alternatively, the display may fulfil a visual function only, and may form part of a personal or laptop computer. The display may be electrically connected to the processor to enable the exchange of data in both directions between the display and the processor and is controlled by the processor to present visual content to a user.

The first device 102 is configured to receive data from the second device 114 and to use that data to output a heart rate variability parameter for a user. The process of outputting a heart rate variability parameter is performed by the plurality of processing modules comprised within the first device 102. Firstly, the input module 104 of the first device 102 is configured to receive a set of heart rate measurements for a user over a period of time, together with data indicating a frequency at which the heart rate measurements have been obtained. The input module may receive the set of heart rate measurements directly from the memory of the second device 114 via connection 118, or alternatively via a network 120 that stores measurement data obtained from the device. The data indicating the frequency at which heart rate measurements have been obtained may comprise the time at which each heart rate measurement was obtained during the period of time. This data may alternatively comprise the number of measurements that were captured during the time period.

The HRV calculator 106 is configured to calculate the heart rate variability parameter over the period of time. The formation of this heart rate variability parameter is described in more detail below.

The uncertainty calculation module 108 is configured to obtain the set of heart rate measurements from the input module and to process the set measurements to estimate whether it is sufficient for calculating a heart rate variability parameter over the period of time. The uncertainty calculation module 108 is further configured to form an uncertainty score representing the result of that estimation. The formation of this uncertainty score is described in more detail below.

The determination module 110 is configured to determine whether the heart rate variability parameter is valid by comparing the uncertainty score formed by the uncertainty calculation module 108 to a predetermined threshold. In some examples, the determination module 110 is configured to determine that the heart rate variability parameter is valid if the uncertainty score is below the predetermined threshold. The predetermined threshold may be defined by one or more values that are stored in the memory of the first device 102. The one or more values of the predetermined threshold may alternatively be stored within an application that is run on the first device 102. In one example, the predetermined threshold is one value. The threshold may be derived from historical data, which may be specific to the user of the first device 102 or to the second device 114 comprising the sensor 116. The threshold may alternatively be derived from historical data obtained from a large number of individuals. In another example, the predetermined threshold may be dependent on the range of heart rate measurements that have been determined. The predetermined threshold may be dependent on the range of values of heart rate that have been received, or on the frequency with which measurements have been taken. The predetermined threshold may be dependent on the time of day at which measurements are taken. The skilled person would understand that the predetermined threshold(s) may be dependent on a number of alternative factors. In one example, the predetermined threshold is 40 ms. It would be appreciated that alternative suitable threshold values may alternatively be selected.

The output module 112 is configured to output, in response to a determination that the heart rate variability parameter calculated by the HRV calculator 106 is valid, the heart rate variability parameter and a representation of the uncertainty score that is formed by the uncertainty module 108 For example, if the uncertainty score is below the predetermined threshold, it may be determined that the set of heart measurements is sufficient for calculating the heart rate variability parameter over the period of time (i.e., valid). Likewise, if the value of the uncertainty score is not below the predetermined threshold, it may be determined that the set of heart measurements is not sufficient for calculating the heart rate variability parameter over the period of time (i.e., not valid). In one example, the set of heart rate measurements is discarded in response to a determination that the heart rate variability parameter is not valid. The discarding of this data advantageously enables memory of the first device 102, second device 114 and/or network 120 to be reserved for data that can be used to obtain reliable heart rate variability parameters. If it is determined that the uncertainty score is the same as or above the predetermined threshold (i.e., the heart rate variability parameter is not valid), then the first device 102 collects a new set of heart rate measurements from the user via the input module 104. The new set of heart rate measurements may be received quasi-simultaneously with the determination that the current set of heart rate measurements is insufficient, or alternatively at a later time. For example, the new set of heart rate measurements may be received on a different day from that on which the current set of heart rate measurements is received.

The heart rate variability parameter and the representation of the uncertainty score outputted by the output module 112 provide a combined indication of whether a user is suffering from cardiovascular disease. Additionally or alternatively, the heart rate variability parameter and the uncertainty score may be used to determine the severity of cardiovascular disease for a user.

The output that is provided by the output module 112 may have a number of different forms. In one example, where the first device is a personal device such as a smartphone, the output comprises demonstrating the heart rate variability and the representation of the uncertainty score on a display of the smartphone. In an alternative example, the output may comprise displaying an alert to a user of the smartphone if the heart rate variability parameter is undesirable. The output may comprise transmitting the alert and/or the heart rate parameter information to an external source, such as a device or network that is accessed by a medical professional. The medical professional may then use the received information to decide on an appropriate course of treatment for the individual. The output may comprise transmitting the heart rate variability parameter to a further calculation unit that determines whether the value of that parameter is indicative of cardiovascular disease.

As described above, the first device 102 is configured to receive heart rate measurements from the second, external device 114 and to calculate a heart rate variability parameter from those measurements. The second device 114 may be a fitness tracker. Commercially available fitness trackers are programmed to measure and output a value of heart rate to a user. However, fitness trackers do not provide the continuous measurement of these heart rate values. Rather, fitness trackers are programmed to provide inconsistent heart rate measurements in order to protect their battery life. That is, the sensors of common fitness trackers are configured to periodically turn on and off to capture discontinuous heart rate measurements.

Fitness trackers are not programmed to measure the duration of heart beats for a user. This is because these trackers, and in particular wrist worn trackers, are heavily affected by the motion of a user whilst they are wearing the device, and so may produce unreliable inter-beat interval readings that is not be suitable for use in the calculation of heart rate variability parameters. An accurate calculation of heart rate variability parameters that are calculated using inter-beat interval length may ideally be calculated from a continuous stream of measurements of inter-beat interval. Such continuous streams of measurements may not be provided by data collected from commercially available wearable devices, which use a low sampling frequency.

Nevertheless, the average inter-beat interval for a user can be predicted from the heart rate data received from a fitness tracker, because the inter-beat interval is inversely correlated to heart rate. That is, the measured heart rate of a user can be used to define the average inter-beat interval over one minute of measurement. For example, if the measured heart rate of a user is 60 bpm, then the inter-beat interval is 60/60=1 second or 1000 ms. SDNN24 is an example of a heart rate variability parameter that is obtained from inter-beat interval measurements, as it is defined as the standard deviation of inter-beat interval collected over a 24 hour period. For the optimal calculation of SDNN24 to be obtained, a continuous stream of inter-beat interval data is preferred.

In order to calculate the inter-beat interval using the average heart rate of a user over a one-minute time period, any recording frequencies that are equivalent to less than one reading per minute are filtered out. Thus, the use of this average heart rate acts as a low pass filter, allowing signals with low frequencies to pass and attenuating signals with higher frequencies.

In some examples, heart rate variability can be observed as a power spectrum. Most of the energy in the power spectrum over a 24 hour period is dominated by ultra-low frequencies. An ultra-low frequency may be considered to have a frequency of less than 0.004 Hz or less than 0.003 Hz or less than 0.002 Hz. That is, ultra-low frequency measurements may have a time period greater than 250 s or greater than 333 s or greater than 500 s. They can therefore be measured using relatively long-term recordings. SDNN24 is a suitable parameter to be observed using ultra-low frequency measurements, because it obtains measurements over a 24 hour period. Moreover, as SDNN24 the standard deviation of the duration of individual heartbeats is the total power of the HRV spectrum, which as discussed above is dominated by the lower end of the spectrum, the contributions made by high frequencies can be discarded without compromising the quality of the estimations. As is known in the state of the art, very low and ultra-low frequency components of heart rate measurements are attributed to the sinoatrial node (SAN), which initiates the electrical impulses that cause contraction of the walls of the heart during systole. See, for example, Rosenberg, A. A., Weiser-Bitoun, I., Billman, G. E. and Yaniv, Y. (2020), “Signatures of the autonomic nervous system and the heart's pacemaker cells in canine electrocardiograms and their applications to humans” Scientific Reports, [online] 10(1), p. 9971. Thus, indicators of cardiovascular disease can be calculated observing only ultra and very low frequencies in the power spectrum of heart rate variability.

Although commercial wrist worn fitness trackers typically measure the average heart rate of a user, and not continuous data such as inter-beat interval, the frequencies of interest (ultra and very low) can still be estimated. This is because periodic heart rate measurements that are obtained from such fitness trackers contain the same information as a continuous stream of inter-beat interval data that has had a low pass filter applied to it.

Another problem associated with commercial fitness trackers is that they provide infrequent heart rate measurements. That is, heart rate may not be measured at regular and consistent time intervals. Instead, a fitness tracker may be programmed to increase the frequency of heart rate measurements at certain times and decrease the frequency of measurements at other times. For example, a fitness tracker may be programmed to increase the frequency of heart rate measurements when a user is performing a physical activity and to decrease the frequency of heart rate measurements when physical activity is not being performed. Additional factors that may result in infrequent heart rate measurements are the programming of a fitness tracker to discard certain measurements because of motion artifacts, such as a user moving a limb to which the tracker is adhered, or because of a removal of the fitness tracker from the limb of a user. In the latter of these examples, the user might remove the tracker during the night so that it can charge. This will result in only heart rate measurements obtained during the day being stored.

As a result of the infrequency of heart rate measurements obtained from fitness trackers, the standard deviation between inter-beat intervals calculated from these measurements can differ, sometimes greatly, from the true value of standard deviation between these intervals. Thus, inaccuracies in heart rate variability parameters such as SDNN24 may be introduced.

To overcome the problems associated with the receipt of infrequently sampled heart rate measurements from a fitness tracker, described herein is an algorithm to assess the quality of received data and determine whether it is suitable for obtaining a heart rate variability parameter such as SDNN24. If the quality of received data is deemed suitable, then the heart rate variability parameter is output. In one example, the algorithm is implemented by the uncertainty module 106 of first device 102. The purpose of the algorithm is to analyse the frequency, or distribution of timing, of heart rate measurements collected over a period of time and determine whether they are sufficient for providing a reliable heart rate variability parameter over the period of time.

The algorithm (e.g., the uncertainty module 106) receives data from the input module 104 of the first device 102, which has been received either directly or indirectly from a second device 114. The received data is data that has been recorded over a pre-defined period of time. In one example, the data comprises heart rate measurements that have been obtained over a 24 hour period. The algorithm may be configured to analyse a subset of heart rate measurements that are deemed suitable for measuring the heart rate variability parameter. As described above, ultra-low frequency components of heart rate measurements may preferably be used to predict a heart rate variability parameter such as SDNN24. Thus, in one example, the algorithm is configured to separate ultra-low frequency components from components at other frequencies. Thus, the components of heart rate measurements that are used to calculate the heart rate variability parameter may be in the ultra-low frequency spectrum. In an alternative example, the components of heart rate measurements that are used to calculate the heart rate variability parameter may be in the low frequency spectrum.

The subset of heart rate measurements may be determined by any suitable filtering function, for example, one that omits instances of heart rate measurements that deviate from neighbouring measurements by greater than a predetermined threshold. The time differences between the points at which measurements that are not omitted were taken may vary due to variation in the underlying measurement frequency and/or due to one or more intervening measurements being omitted following filtering. The result is a non-empty set of observation points. Each measurement point represents a heart rate frequency/period measurement and the time at which that measurement was taken.

Next, the algorithm (e.g., the uncertainty module 106) analyses the set of observation points to establish a fit to that set of a curve of varying heart rate/frequency over time of measurement where such a curve has a frequency or a frequency component below a predetermined threshold, such as 0.003 Hz. The system may seek a best fit to a time average of the observed heart rate/frequency values where that average is over a time such as 60 to 120 s. In a second example, the subset comprises linear functions.

It is possible to compute SDNN24 from only low frequency components of the inter-beat interval spectrum over a maximum period of 24 hours, because the SAN activity produces a spectrum with characteristic 1/f shape. Once the best fit of a curve having a component less than the predetermined frequency has been estimated, SDNN24 can be estimated as the reciprocal of the frequency of that component. A least square fit on a plausible function space may be used to estimate the best fit. The least squares method is a statistical procedure to find the best fit for a set of data points by minimizing the sum of the offsets or residuals of points from the plotted curve. As the SAN activity produces a spectrum with a 1/f shape, it is the power values at lower frequencies that are of higher importance when calculating the heart rate variability parameter for a user. It is therefore important to monitor power values at low and ultralow frequencies. Thus, the use of a low pass filter to attenuate signals with higher frequencies does not have a large effect on heart rate variability calculations, as power values at higher frequencies are of lesser importance.

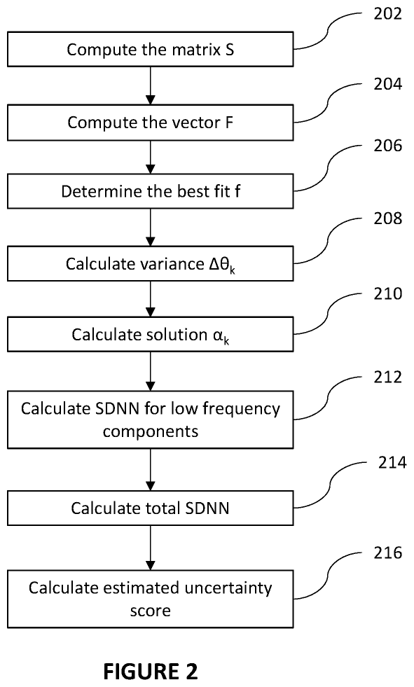

FIG. 2 illustrates a method for calculating a heart rate variability parameter and an uncertainty score for a user. In this example, the heart rate variability parameter is SDNN24. The steps that are performed using this method are detailed below:

At step 202, a matrix S is computed using the following equation:

$${S}_{k , l} = {S}_{k - l} = {\sum }_{j} {w}_{j} {e}^{i ( k - l ) {x}_{j}}$$

where, xⱼ is the time of the j^{th} heart rate measurement and wⱼ is the weight assigned to the j^{th} heart rate measurement. Assuming that heart rate is measured over 1 minute of inter-beat intervals, the heart rate measurements themselves can be used as weights.

At step 204, a vector F is computed using the following equation:

$${F}_{k} = {\sum }_{j} {w}_{j} {y}_{j} {e}^{ikj}$$

where, yⱼ is the reciprocal of the j^{th} measure of heart rate.

At step 206, the best fit f can be determined from S and F by solving the following linear system:

S·f=F

At step 208, Δθk is calculated. Δθk is the variance of the k^{th} value of a parameter θ, and is calculated using the following equation:

$$Δ {θ}_{k} = \frac{{Δ}_{0}}{\sqrt{{λ}_{k}}}$$

where, λₖ is the eigenvalue of S corresponding to the normalised eigenvector vₖ, and Δ₀ is a parameter expressing how much information one heart rate measurement adds to the module. Δ₀ is a constant that may be derived during construction of the algorithm. In some examples, Δ₀ expresses how much information each heart rate measurement contributes to the uncertainty score. In one example, Δ₀ is 30000. It would be appreciated that alternative values of Δ₀ can be used.

At step 210, αₖ is calculated. by solving the system Vαₖ=βₖ, where βₖ is the vector [1, 1, 0, 1, 1]. The purpose of βₖ is to take all the non-zero Fourier components. αₖ is the solution of a system that indicates which linear combination of eigenvectors will produce βₖ. V is the plausible function space, which is a subspace of the total function space that is defined by the total number of heart rate measurements comprised within a set of heart rate measurements. That is, the plausible function space V comprises a subset of heart rate measurements from the set of heart rate measurements.

At step 212, an estimated SDNN24 value for the low frequency components is calculated using the following equation:

$${SDNN}_{L F} = 60000 \sqrt{{\sum }_{k = - 2}^{2} {\vert {f}_{k} \vert}^{2} {β}_{k}}$$

In an alternative example, the SDNN24 value for the ultra-low frequency components may be calculated.

At step 214, an estimated value of SDNN24 is calculated using the following equation:

SDNNₑₛₜ=q+mSDNN_{LF}

In the example where an SDNN24 value for the ultra-low frequency components is be calculated, SDNN_{LF}=SDNN_{ULF}. In the above equation, q and m are constants that may be derived during construction of the algorithm. In one example, the value of q is 29 ms and the value of m is 1. It would be appreciated that alternative values of q and m may alternatively be used. The above algorithm can be used by HRV calculator 106 to calculate the heart rate variability parameter.

At step 216, an estimated or predicted uncertainty score for the SDNN24 parameter is derived. This uncertainty score is derived using the following equation:

$$Δ S D N {N}_{e s t} = \frac{m}{S D N {N}_{L F}} {\sum }_{k = - 2}^{2} {α}_{k} Δ {θ}_{k}$$

The above algorithm can be used by the uncertainty module 108 to calculate an uncertainty score representing the result of an estimation of whether a set of heart rate measurements is sufficient for the heart rate variability parameter (i.e. SDNN24) to be reliably calculated.

The algorithms described above can estimate SDNN24 and its associated uncertainty score over punctuated data sets: that is data sets whose heart rate measurements have been measured at irregular intervals. Such irregularity may result from a range of events that cause measurements not to be taken or cause measured data to be discarded as potentially unreliable. In algorithms such as those described above, predetermined constants can be used to adjust the results so as to conform to underlying results. Examples of such constants include q, m and AO as discussed above. Suitable values for such constants can be estimated by receiving a set of regularly measured heart rate values, estimating the SDNN24 for that set, deleting selected values from that set so as to form a reduced data set, estimating SDNN24 over that reduced data set using candidate values for the constants, and adapting the candidate values in dependence on a comparison between the SDNN24 estimated over the full data set and the SDNN24 estimated over the reduced data set. Values for deletion may be selected using any suitable mechanism.

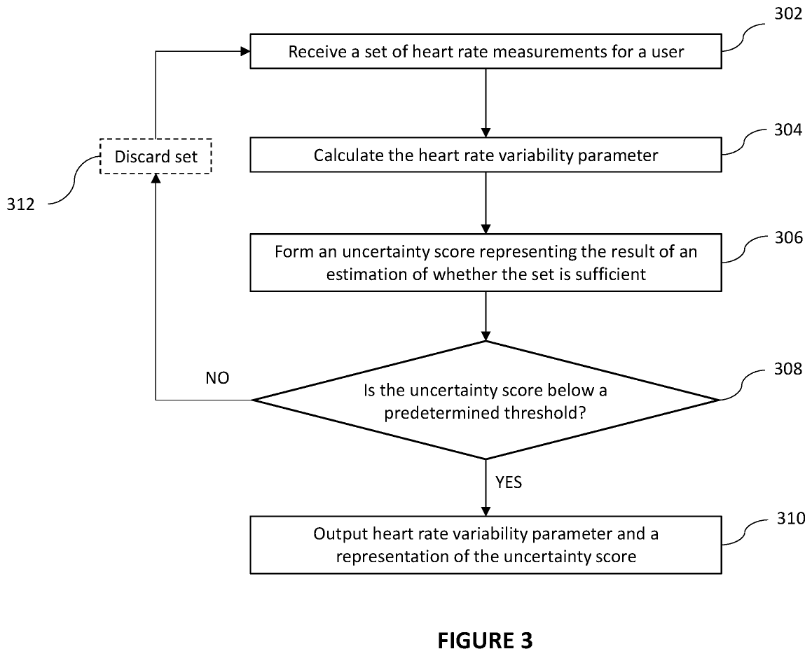

FIG. 3 illustrates a method for monitoring the heart rate variability of a user, using the first device illustrated in FIG. 1 and the corresponding algorithms described above. The method is initiated at step 302, when a set of heart rate measurements is received from a user over a period of time. The heart rate measurements are received either directly from the second device 114 via connection 118, or indirectly from the second device via the network 120. As mentioned above, the network 120 is configured to store data recorded by the second device 114 (e.g., via a cloud-based memory). The set of heart rate measurements may be received over a 24 hour period. For example, the set of heart rate measurements for one day may be received simultaneously. The set of heart rate measurements may alternatively be received by the first device 102 periodically querying the second device 114, or the network 120 to which the second device 114 is connected. In addition to the heart rate measurement values, the set of heart rate measurements comprises data indicating the frequency at which the heart rate measurements have been obtained. This data may comprise the time at which each heart rate measurement was obtained during the period of time. This data may alternatively comprise the number of measurements that were captured during the time period. As described above, the set of heart rate measurements comprise measurements obtained at infrequent intervals.

At step 304, once the set of heart rate measurements for the user has been received by the first device 102, the heart rate variability parameter is calculated. The heart rate variability parameter to be calculated may be the standard deviation of the inter-beat interval of sinus beats. The standard deviation of the inter-beat interval of sinus beats may be calculated over a 24 hour time period. This standard deviation is otherwise referred to as SDNN24. It would be appreciated that alternative heart rate variability parameters may be used in place of SDNN24.

At step 306, the set of heart rate measurements are processed by the first device 102 to estimate whether that set is sufficient for calculating a heart rate variability parameter over the period of time. An uncertainty score is formed representing the result of that estimation. In other words, at step 306 the quality of the set of heart rate measurements is analysed to determine whether it is sufficient to predict a reliable estimate of the heart rate variability parameter. This estimation may be calculated using the method illustrated in FIG. 2.

At step 308, it is determined whether the heart rate variability parameter is valid by comparing the uncertainty score to a predetermined threshold. In one example, the heart rate variability parameter is determined to be valid if the uncertainty score is below the predetermined threshold. The predetermined threshold may be defined by one or more values that are stored in the memory of the first device 102. The one or more values of the predetermined threshold may alternatively be stored within an application that is run on the first device.

If it is determined that the heart rate variability parameter is valid (e.g., the uncertainty score is below the predetermined threshold), then the first device proceeds at step 310 to output the heart rate variability parameter. A representation of the uncertainty score is also output. That is, if the uncertainty score is below the predetermined threshold, then it may be determined that the set of heart rate measurements is sufficient for the heart rate variability parameter and the representation of the uncertainty score to be reliably output. In one example, the predetermined threshold corresponds to an amount of time (e.g., 40 ms). However, it should be appreciated that alternative suitable threshold types and values may be selected. For example, the predetermined threshold may represent a grade, ranking, or percentage (e.g., B+, 90%, etc.) that the uncertainty score is compared to. In such examples, the heart rate variability parameter may be determined to be valid if the uncertainty score is above the predetermined threshold.

The output may comprise sending the heart rate variability parameter and the representation of the uncertainty score to further systems which perform further analyses and action interventions should the parameter level drop below a certain threshold, with a small enough uncertainty. That is, the parameter may be compared to a baseline value to determine whether a user is experiencing an atypical heart rate variability characteristic. The baseline may be specific to a user of the first device 102 and may be based on historically recorded data obtained from that user. For example, if a user has a baseline value for SDNN24 of 100 ms, but the output from the device 102 indicates that the prediction of SDNN24 is 60 ms with an uncertainty score of 15 msec, then it may be determined that the user is at risk of or suffering from cardiovascular disease. The output may be provided to the medical professional, and the medical professional may decide to initiate a course of treatment as a result of receiving the output. The output may comprise transmitting the heart rate variability parameter (e.g., via the network 120) to a further calculation unit that determines whether the value of that parameter is indicative of cardiovascular disease. The output may additionally or alternatively comprise demonstrating the heart rate variability and the representation of the uncertainty score on a display of the first device 102.

In some examples, if it is determined that the heart rate variability parameter is not valid e.g., the uncertainty score is the same as or above the predetermined threshold), then the first device 102 may proceed to step 312 in which the set of heart rate measurement data is discarded. This is an optional step and is illustrated as such in FIG. 3 by the dashed box. In an alternative embodiment the data is not discarded but is instead stored within the memory of the first device 102 (or a cloud-based memory via the network 120) so that it may be used for subsequent calculations. The first device 102 then reverts to step 302 where a new set of heart rate measurements for a user is received.

Embodiments of the invention may include any individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. Aspects of the present invention may include any such individual feature or combination of features. In view of the foregoing description it will be evident to a person skilled in the art that various modifications may be made within the scope of the invention.

What is claimed is:

1. A device for predicting a heart rate variability parameter for a user, the device comprising one or more processing modules and being configured to:
receive a set of heart rate measurements for a user obtained over a period of time, together with data indicating a time at which each heart rate measurement was obtained during the period of time;
process the set of heart rate measurements to calculate a heart rate variability parameter over the period of time;
further process the set of heart rate measurements to estimate whether the set is sufficient for calculating the heart rate variability parameter over the period of time, and to form an uncertainty score representing the result of that estimation;
determine whether the heart rate variability parameter is valid by comparing the uncertainty score to a predetermined threshold; and
output, in response to a determination that the heart rate variability parameter is valid, the heart rate variability parameter and a representation of the uncertainty score.

2. A device as claimed in claim 1, wherein the set of heart rate measurements is received from a second device that is external to the first device, the second device comprising a sensor for recording a plurality of heart rate measurements over time.

3. A device as claimed in claim 2, wherein the second device is a wrist-worn fitness tracker.

4. A device as claimed in claim 2 or claim 3, wherein the set of heart rate measurements is received directly from the second device via a wired or wireless connection.

5. A device as claimed in claim 2 or claim 3, wherein the set of heart rate measurements is received via a network that stores data recorded by the second device.

6. A device as claimed in any preceding claim, wherein the heart rate variability parameter is determined to be valid if the uncertainty score is below the predetermined threshold.

7. A device as claimed in any preceding claim, wherein the set of heart rate measurements is discarded if it is determined that the heart rate variability parameter is not valid.

8. A device as claimed in any preceding claim, wherein the heart rate variability parameter is the standard deviation of the inter-beat interval of sinus beats.

9. A device as claimed in claim 8, wherein the period of time is a 24 hour time period.

10. A device as claimed in any preceding claim, wherein the heart rate measurements that are used to calculate the heart rate variability parameter are in the ultra-low frequency spectrum.

11. A device as claimed in any preceding claim, wherein the heart rate variability parameter is output to a further calculation unit that determines whether the value of that parameter is indicative of cardiovascular disease.

12. A device as claimed in any preceding claim, wherein the output comprises demonstrating the heart rate variability and the representation of the uncertainty score on a display of the first device.

13. A device as claimed in claim 9, wherein an estimated value of the heart rate variability parameter is calculated using the following calculation:
SDNNₑₛₜ=q+mSDNN_{LF}
wherein SDNN_{LF} is defined by:
$$S D N {N}_{L F} = 60000 \sqrt{{\sum }_{k = - 2}^{2} {\vert {f}_{k} \vert}^{2} {β}_{k}}$$
wherein fₖ is a calculated best fit parameter and βₖ is a vector defined as [1, 1, 0, 1, 1].

14. A device as claimed in claim 13, wherein the uncertainty score is formed using the following calculation:
$$Δ S D N {N}_{e s t} = \frac{m}{S D N {N}_{L F}} {\sum }_{k = - 2}^{2} {α}_{k} Δ {θ}_{k}$$
wherein αₖ is a solution of a system that indicates which linear combination of eigenvectors will produce the vector βₖ and Δθₖ is a variance of the k^{th} value of a parameter θ, and is defined by:
$$Δ {θ}_{k} = \frac{{Δ}_{0}}{\sqrt{{λ}_{k}}}$$
wherein λₖ is an eigenvalue of a matrix S corresponding to a normalised eigenvector vₖ, and Δ₀ is a constant expressing how much information each heart rate measurement contributed to the uncertainty score.

15. A method for predicting a heart rate variability parameter for a user using a first device, the method comprising:
receiving a set of heart rate measurements for a user obtained over a period of time, together with data indicating a time at which each heart rate measurements was obtained during the period of time;
processing the set of heart rate measurements to calculate a heart rate variability parameter over the period of time;
processing the set of heart rate measurements to estimate whether the set is sufficient for calculating the heart rate variability parameter over the period of time and forming an uncertainty score representing the result of that estimation;
determining whether the heart rate variability parameter is valid by comparing the uncertainty score to a predetermined threshold; and
outputting, in response to a determination that the heart rate variability parameter is valid, the heart rate variability parameter and a representation of the uncertainty score.
